(19) United States
(12) Patent Application Publication
(10) Pub. No.: US 2026/0055359 A1
(43) Pub. Date: Feb. 26, 2026

(54) AUTOMATED SYSTEM FOR BIOPROCESSING

(71) Applicant: CELLUAR ORIGINS LIMITED, Melbourn (GB)

(72) Inventors: Dan Strange, Melbourn (GB); Leo Steenson, Melbourn (GB); Paul Crisp, Melbourn (GB)

(21) Appl. No.: 19/107,440

(22) PCT Filed: Aug. 31, 2023
(86) PCT No.: PCT/GB2023/052259

§ 371 Date: Feb. 27, 2025

(30) Foreign Application Priority Data
Aug. 31, 2022 (GB) 2212684.1

(51) Int. Cl. C12M 1/36; C12M 1/00; C12M 1/12

(52) U.S. Cl. CPC C12M 41/48; C12M 23/28; C12M 23/40; C12M 23/50; C12M 37/04; C12M 41/14

(57) ABSTRACT

An automated system for performing bioprocessing, comprising: an incubation system configured to accommodate at least one incubation chamber for incubating a cell culture chamber having a flexible tube connected fluidly thereto; and a storage system configured to store at least one fluid-containing consumable having a tube connected fluidly thereto; wherein the incubation system and storage system are together arranged to enable a robotic device to manipulate a tube weld between said flexible tube of the incubated cell culture chamber and said tube of the stored consumable, whereby said tube weld forms an aseptic fluid connection between the cell culture chamber and the consumable such that a closed system is maintained.

### FIELD OF THE INVENTION

The present disclosure relates to automated bioprocessing. More specifically, the disclosure relates to an automated system, which may be configured to perform multiple bioprocessing steps or operations in parallel while maintaining a closed system.

### BACKGROUND

Therapeutics are increasingly using cells rather than small molecules as the starting point. The approaches to manufacturing these products are rapidly evolving to keep up with constantly emerging new therapies. In recent years, there has been an increased use of a number of new classes of cell therapies. One class is autologous cell therapies.

Autologous cell therapies are a promising class of therapy, which have significant clinical and commercial potential ranging from treating cancer to fixing genetic defects. These therapies involve taking cells from a patient, manipulating the cells over the course of days to weeks, and re-introducing the cells back into that patient's body to produce a therapeutic effect. The steps taken during autologous cell therapies are often complex; for example, a typical CAR-T process may involve a sequence of steps starting with a cryopreserved leukopak, thawing, washing to remove DMSO, enrichment of T cells, activation, transduction, expansion, concentration, formulation fill finish into an IV bag, and cryopreservation, with several other intermediate washing steps.

Bioprocessing systems have been developed for carrying out the above steps. Within such a bioprocessing system, multiple different consumables (e.g. fluid-containing bags) may be required to supply media, reagents and/or cell material to an expansion chamber (e.g. a cell culture chamber), for example, throughout the cell therapy process. Similarly, one or more consumables (e.g. an output or waste bag consumable) may receive fluid samples from the expansion chamber.

The term “consumable” may therefore be used to refer to a “single-use” element or component of the system.

Some of the processes discussed above such as activation, transduction and expansion may be carried out within a cell culture chamber, which may be incubated in a bioreactor (i.e. an “incubation chamber”). During one or more of those processes, various functions need to be performed, such as perfusion, gas and nutrient transport, reagent mixing, media conditioning and heat exchange.

However, due to the need to maintain a closed system when handling the cells, reagents and other fluids, conventional consumables (e.g. media bags) include a complicated network of external flexible tubing for interfacing with the incubation chamber. This network of flexible tubing can be difficult to handle, particularly by an automated means such as a robotic device. Additionally, it can be complicated to store such consumables in a manner in which they can be readily accessed and used in a space efficient way within a bioprocessing system.

The ability to store and operate multiple bioreactors simultaneously, including supplying the necessary fluids from consumables to each bioreactor, when required, can be challenging due to the need for specialised equipment to interface with each consumable, and it can therefore be difficult and expensive to run several incubation processes in parallel. Furthermore, a typical bioprocessing system has limited capacity for bioreactors, and the number of expansion chambers (e.g. cell culture chambers) that may be accommodated at any one time is limited accordingly.

### SUMMARY OF INVENTION

Described herein is an automated system for performing bioprocessing, comprising: an incubation system configured to accommodate at least one incubation chamber for incubating a cell culture chamber having a flexible tube connected fluidly thereto; and a storage system configured to store at least one fluid-containing consumable having a tube connected fluidly thereto; wherein the incubation system and storage system are together arranged to enable a robotic device to manipulate a tube weld between said flexible tube of the incubated cell culture chamber and said tube of the stored consumable, whereby said tube weld forms an aseptic fluid connection between the cell culture chamber and the consumable such that a closed system is maintained.

During use, one or more incubation chambers and/or one or more fluid-containing consumables may be accommodated or stored by the automated system, though it will be appreciated that these chambers and consumables are removable and replaceable from the automated system. The incubation system may have one or more features that retain an incubation chamber in a predetermined position in the incubation system, such as at least one incubator base with dimensions corresponding to an incubation chamber. The storage system may comprise a plurality of consumable holding positions or slots, each configured to receive and store a consumable. Each slot may be configured to receive a cartridge containing a consumable. Each slot may have one or more engagement features to releasably retain the consumable (e.g., in a respective cartridge) within the slot.

The system may be configured as a bioreactor system for a bioprocessing system. The system may therefore form part of a larger bioprocessing system.

The incubation system may be further configured to store in each incubation chamber a cell culture chamber with its flexible tube in a predetermined orientation, optionally wherein a portion of the flexible tube is held external of the incubation chamber, for example by one or more tube clips. In this way, the tube may be manipulated to connect it to different consumables without opening the incubator and risk affecting the temperature of the incubator.

The storage system may be further configured to store each consumable with its a tube held in a predetermined orientation, for example by one or more tube clips.

The incubation system may be configured to accommodate a single incubation chamber. Alternatively, the incubation system may be configured to accommodate a plurality of incubation chambers, each arranged to incubate an individual cell culture chamber in physical isolation from a neighbouring incubation chamber.

The incubation system may comprise a single incubation chamber for a single cell culture chamber, or a single incubation for a plurality of cell culture chambers. Alternatively, or additionally, the incubation system may comprise a plurality of incubation chambers, each configured to receive a single cell culture chamber.

Each incubation chamber may be temperature-controlled. Each incubation chamber may be gas-controlled.

At least part of the storage system may be arranged to be elevated relative to the incubation system. Optionally, at least part of the storage system may be below the incubation system; in this way, backflow of waste from the waste containers to the incubation section is inhibited.

The storage system may comprise a rack arrangement having one or more apertures for receiving the consumables.

The system may further comprise a cell analysis unit, wherein the at least one robotic device is further configured to manipulate a fluid connection between the cell analysis unit and an incubated cell culture chamber.

The cell analysis unit may have at least one tube connected fluidly thereto, and the at least one robotic device may be further configured to manipulate a fluid connection between said tube fluidly connected to the cell analysis unit and a tube fluidly connected to an incubated cell culture chamber. The cell analysis unit (or “cell analyser”) may be a cell counter, a cytometer, or any other means of cell analysis or media analysis.

Alternatively, the system may take a sample by welding on a vial where one end has a septum and the other has a tube, and then the system pumps a sample into the vial and then disconnects the tube. The robot the takes the vial to the cell analysis unit which then punctures the septum with a needle, since there is no requirement for the sample analysis process to be closed.

One or more processing parameters of the system may be configured to be adjusted based on measurements by the cell analysis unit.

The system may further comprise an automated processing station at which processes and operations for bioprocessing are automated, for example using one or more robotic devices. Said automated processing station is preferably configured as a stand-alone processing station. The automated processing station may include a support surface or platform configured to form part of the incubation system. The storage system may be in an elevated position relative to the support surface or platform.

The automated system may comprise the robotic device. At least one robotic device may also be provided on the processing station. In other words, the robotic device that manipulates the tube weld is part of the processing station.

The system (e.g., the processing station) may further comprise a movement system for the at least one robotic device that is configured to move the robotic device relative to at least one of the incubation system and the storage system.

The movement system may comprise at least one rail (e.g., on the processing station) to which the at least one robotic device is movably mounted, and a drive system for moving the robotic device along the rail to one or more predetermined positions.

Alternatively, the at least one robotic device may be separate to the processing station. According to another aspect of the present invention, there is provided a bioprocessing system comprising the automated processing station described above and herein, and a separate robotic device configured to move independently relative to the automated processing station. In other words, the robotic device that manipulates the tube weld may be the separate robotic device.

For example, the (at least one) robotic device may perform other functions in the bioprocessing system as well as manipulation of sterile tube welds between the flexible tubes of the processing station. Optionally, a robotic device may also be provided as part of the processing station (e.g., in addition to at least one separate robotic device that may move independently to the processing station).

The robotic device may be configured to manipulate the fluid connection whereby to create a tube weld between the flexible tube of an incubated cell culture chamber and the flexible tube of a stored consumable for the transfer of fluid therethrough, said tube weld being a sterile tube weld formed between free ends of the respective tubes.

The robotic device may be configured to manipulate the fluid connection whereby to disconnect the flexible tube of an incubated cell culture chamber and the flexible tube of a stored consumable.

The robotic device may be configured to manipulate the fluid connection whereby to seal one or both of said flexible tubes, preferably prior to a disconnection of the fluid connection.

The robotic device may be configured to manipulate the fluid connection whereby to pump fluid between a consumable and an incubated cell culture chamber via said fluid connection.

The robotic device may be further configured to pump fluid by applying a peristaltic pumping action to at least one of the flexible tubes forming the fluid connection.

The robotic device may be configured to manipulate the fluid connection whereby to engage and/or position the respective at least one flexible tube of an incubated cell culture chamber and at least one flexible tube of a stored consumable relative to one another to form the fluid connection.

The at least one robotic device may be further configured to manipulate a fluid connection between a first incubated cell culture chamber and a second incubated cell culture chamber.

The robotic device may be configured to use interchangeable end effectors (e.g., for a robotic arm of the robotic device), the system further comprising an end effector storage system for storing one or more interchangeable end effectors for use by the robotic device.

The robotic device may comprise an end effector on a robotic arm and the robotic device may further comprise at least one of a probe or sensor located on the end effector. The probe or sensor may be a Raman probe, an optical probe, a microscope, and/or any other suitable type of probe or sensor. It will be appreciated that such probes are not required to directly contact the cells in order to collect data, thereby allowing the cells to be monitored while maintaining a closed system.

The system may further comprise a movement system for the at least one robotic device that is configured to move the robotic device relative to at least one of the incubation system and the storage system.

The movement system may comprise at least one rail to which the at least one robotic device is movably mounted, and a drive system for moving the robotic device along the rail to one or more predetermined positions.

The at least one robotic device may comprise a first robotic device and a second robotic device.

The first robotic device may be configured to create a sterile tube weld, and the second robotic device may be configured to perform at least one of the following: manipulate the fluid connection whereby to pump fluid therethrough or seal tubes, preferably prior to a disconnection of the fluid connection. In this way, the second robotic device may be used to seal or pump fluid away from the weld; in the event that there is a weld failure, the first robotic device may be used to reweld the tubes.

The system may further comprise a controller for controlling a sequence of automated operations of the system. The controller may be configured to control the automated sequence of operations according to one or more predetermined workflows. Preferably, the one or more predetermined workflows are reconfigurable workflows.

The controller may be configured to schedule automatically a sequence of actions to be followed by the system. The system may further comprise a user interface configured to enable a user to programme a pre-determined workflow for the system to follow. The controller may be configured to simulate the automated sequence of operation prior to the system performing said sequence. The system may be attached to a computer network so that one or more systems can be monitored and controlled remotely. The one or more systems may be controlled as a group of processing stations, or as subsystems within a more complex system.

The system may further comprise a fluid agitation system for agitating fluid contained within an incubated cell culture chamber. The fluid agitation system may be configured to agitate fluid within the or each incubated cell culture chamber prior to the transfer of fluid from a cell culture chamber to an output container and/or a sample container.

The at least one robotic device may be configured to manipulate the fluid connection whereby to facilitate the transfer of fluid from at least one incubated cell culture chamber to a waste container prior to agitation of the incubated cell culture chamber by the fluid agitation system.

The storage system may comprise a refrigeration or heating unit for regulating the temperature of one or more consumable.

The system may comprise at least one of a probe or sensor located on an end effector of the robotic arm.

The system may further comprise a tube supply device arranged to provide supplementary tubing for at least one of a cell culture chamber or a consumable, preferably wherein the device comprises a reel of tube supply.

The system may further comprise means for identifying an identification mark on at least one of the following: consumables, tubes, cell culture chambers. The identification mark may optical or non-optical. The identification mark may be a barcode, QR code, RFID or NFC code.

The system may comprise a self-contained processing station arranged to facilitate transfer of fluid from a stored consumable to an incubated cell culture chamber at the processing station.

Also described herein is a bioprocessing system comprising a plurality of automated processing systems described above and herein.

Also described herein is a method comprising performing (automated) bioprocessing using a system as described above and herein.

As used herein, the term “processing station” preferably connotes an apparatus, work station, unit, module or “stand-alone” system, which may form part of a larger system, such as an automated bioprocessing system. As used herein, the term “standalone” preferably connotes that the processing station is able to operate independently as part of a larger system due to it being able to provide the necessary consumables, incubation chambers, cell culture chambers and tubing required to perform the necessary processes and operations.

As used herein, the term “automated” preferably connotes a process or operation that may be performed entirely without human intervention once started. Preferably, the process or operation may also start and finish without human intervention, other than to programme the equipment that performs the operation or process.

As used herein, the term “closed system” preferably connotes that there is no contamination to or from the surrounding environment (e.g. transfer of material from the cell culture chamber to the surrounding environment, and vice versa) during the operations or processes performed at the processing station. As used herein, the term “closed system” may further connote a “functionally” closed system, or more preferably a fully closed system, where a physical barrier is maintained between the surroundings and the contents of the cell culture chambers and consumables. In a functionally closed system, air may be supplied to the system via a sterilising air filter, for example, which while not fully closed, is considered to be a sufficiently “closed system” to prevent contamination.

As used herein, the term “tube welder” refers to any device that is configured to join (i.e. weld) a first tube to a second such tube (preferably at their free ends), thereby providing an aseptic (and preferably closed) fluid connection between the tubes. Briefly, a tube welder may comprise a first clamping unit and a second clamping unit. Each clamping unit may comprise a pair of jaws movable between an open position for receiving a flexible tube therebetween, and a closed position for clamping a received tube. The clamping units may be located on a robotic arm. When a tube is clamped, the flexible tube is pinched shut, preferably inhibiting any flow of fluid therethrough.

The clamping units may be operated to grip the tubes without clamping them shut; this may enable the tubes to be engaged and positioned without inhibiting flow of fluid. When a first tube is clamped by the first clamping unit and a second tube is clamped by the second clamping unit, a cutting blade may be heated and moved to intersect a clamped portion of both of the tubes. This cuts each tube into an upstream portion leading to a respective consumable, and a downstream portion that previously led to a closed end of the tube. Heat from the cutting blade is transferred to the tubes, thereby at least partially melting each flexible tube at the newly formed cut ends. Subsequently, the clamping units are moved so as to locate the upstream portions tubes adjacent to each other. The downstream portions may be discarded. Once the blade is removed, the upstream portions may be pressed into each other, thereby welding the tubes together to form a single tube. The joint may be referred to as a butt-weld. At this stage, the joint between the tubes may remain pinched shut; a pinch release mechanism may be operated to remove the pinched portion, thereby establishing a fluidic path through the joined tubes. A visual or mechanical quality control (QC) means may also be provided to confirm that the weld has been successfully welded together.

As used herein, the term “peristaltic pump” may refer to a rotary peristaltic pump or a linear peristaltic pump. A peristaltic pump is configured to compress a portion of the flexible tube, and then move the compressed portion along the length of the tube in a pumping direction, thereby forcing fluid through the tube. Advantageously, the peristaltic pump may allow fluid to be rapidly pumped through the tubes, with minimal wear to the tubes and minimal chance of contamination.

As used herein, the term “consumable” preferably connotes a container, such as a bag holding a fluid containing, for example, cellular samples (or cell material), reagents or fluids, which may be intended to be processed at the processing station as part of a cell therapy process, for example. A cell suspension bag is therefore an example of a consumable in the context of the present disclosure. Other types of consumable include media bags, sample bags, intermediate process bags, waste bags and output bags. In the context of the present disclosure, each consumable has fluidly connected thereto an “upstream” end portion of a tube that provides a fluid conduit to a “downstream” (opposite) end portion of the tube, which is fluidly sealed by a portion of the tube being pinched closed when not connected to another such (second) tube.

As used herein, the term “expansion chamber” is a specific type of cell culture chamber. Accordingly, the term “cell culture chamber” may be interchanged with “expansion chamber” within the scope of the present disclosure, noting that the processing station may be used to perform processes and operations described herein (e.g. fluidly connecting a consumable) on different types of cell culture chamber, of which an expansion chamber is simply one example.

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that embodiments are described herein purely by way of example, and modifications of detail can be made within the scope of the disclosure. Furthermore, as used herein, and “means plus function” features may be expressed alternatively in terms of their corresponding structure.

### BRIEF DESCRIPTION OF DRAWINGS

One or more embodiments will now be described, purely by way of example, with reference to the accompanying figures, in which:

FIG. 1 shows a first embodiment of an automated processing system;

FIG. 2a shows an example of an incubation chamber for use with the processing system of FIG. 1, with FIGS. 2b and 2b showing components of the incubation chamber in more detail;

FIG. 3 shows an example of a consumable holder for use with the processing system of FIG. 1;

FIGS. 4A and 4B show a second embodiment an automated processing system;

FIGS. 5A and 5B shows a third embodiment of an automated processing system; and

FIG. 6 shows an example of a consumable holder for use with the processing system of FIGS. 4 and 5.

### DETAILED DESCRIPTION

In the following description and accompanying drawings, corresponding features may preferably be identified using corresponding reference numerals to avoid the need to describe said common features in detail for each and every embodiment.

In general terms, an automated processing system is described herein, which may be configured to perform multiple processes or operations in parallel while maintaining a closed system. The processing system may form part of a larger automated bioprocessing system. Such an automated system may be configured to perform a cell therapy process, for example. Operations and steps that have traditionally been performed by a human operator are automated to improve accuracy and precision of the operations and processes, and hence repeatability. More specifically, the automated processing system may facilitate certain operations and processes in parallel; in particular, the incubation of a cell culture chamber and the supply of fluids containing reagents (or other materials, such as cell material) to the cell culture chamber from a consumable, while maintaining a closed system. In preferred embodiments, which will be described in more detail further on, the automated processing system may comprise a processing station (e.g. an “apparatus”) at which such processes and operations are automated using one or more robotic devices.

In preferred embodiments, a closed system may be maintained by using an automated robotic device to manipulate (e.g. create) fluid connections between the fluid-containing consumable and the incubated cell culture chamber at the bioprocessing station. The robotic device may form aseptic fluid connections between other consumables; for example, the robotic device may manipulate a fluid connection between two incubated cell culture chambers, and/or may manipulate a fluid connection between a cell culture chamber and an analysis chamber (or any other suitable container or consumable). The fluid connections created by the automated robotic device are tube welds formed between (the free ends) of portions of (typically) flexible tubes that are fluidly connected to each of the consumable and the incubation (typically) chamber/cell culture chamber.

More specifically, the tube welds that are formed by the robotic device are sterile tube welds, which may be created by a tube welding apparatus or “tube welder”. This may be achieved by the robotic device comprising a robotic arm having mounted to it an end effector configured as a “tube welder”. For example, the end effector may be configured to engage with an end portion of a tube that is fluidly connected to a consumable, clamp a portion of the tube at a position along the tube spaced from the end of the tube to form a pinched portion that fluidly seals the tube (and hence the consumable), remove the existing (free) end of the tube to form a new (free) end of the tube that has not previously contacted another such tube, and then bring together the new end of the tube with a correspondingly formed new end of another such tube that is in fluid connection with the cell culture chamber, forming a fluid connected between the consumable and cell culture chamber by performing a tube weld to join together the ends of the two tubes.

In other words, the robotic device may be configured to manipulate a fluid connection. To do so, it may engage and/or position (an end portion of) a flexible tube of an incubated cell culture chamber and (an end portion of) a flexible tube of a stored consumable relative to one another to form the fluid connection, which is preferably created by the robotic device further performing a tube weld to join the respective end portions of the two tubes.

Once fluidly connected, fluid from a consumable can be transferred into the cell culture chamber. This may be achieved, for example, by a pumping mechanism (or means) configured to apply a peristaltic pumping action to the fluidly connected tubes to urge the fluid through them from the consumable to the cell culture chamber, the mechanism comprising a plurality of moveable pressing elements arranged to be brought into contact with a portion of tube sequentially, which movement may be repeated. In other words, the pumping mechanism may comprise a peristaltic pump. The pumping mechanism may be provided by a robotic device comprising a robotic arm having mounted to it an end effector configured as such a pumping mechanism. Alternatively, a pumping mechanism may be provided at the processing station, the mechanism arranged to receive a portion of the fluidly connected tubes and apply a pumping action thereto to urge fluid through the tubes from the consumable to the cell culture chamber.

Once a fluid transfer has taken place between a consumable and a cell culture chamber, the fluid connection formed between their respective flexible tubes may be disconnected. When the tubes are disconnected, they are sealed so as to maintain a closed system. The tubes are preferably sealed prior to the disconnection of the fluid connection. The disconnection and sealing may be achieved using the tube welder (e.g. which is further configured to seal and disconnect the tubes), or a separate tube sealer may be used for this purpose. The tube sealer may be provided on the same robotic device as the tube welder and/or the peristaltic pump. The tube sealer may use heat and/or RF radiation to seal the flexible tubes.

As will be discussed further on, one or more robotic device(s) may be mounted to the processing station, or they may be provided as part of an autonomous mobile (“manipulator”) unit/robot device that is configured to move autonomously around a bioprocessing system (e.g. a system provided in an enclosed space) in which one or more such processing stations are situated, and to attend the or each processing station when manipulation of a fluid connection is required. Multiple robotic devices/arms may be provided, each having mounted thereto an end effector configured to perform a specific function, or a single robotic device/arm may be configured to have interchangeable end effectors, which may be stored at the processing station and/or on the autonomous mobile unit.

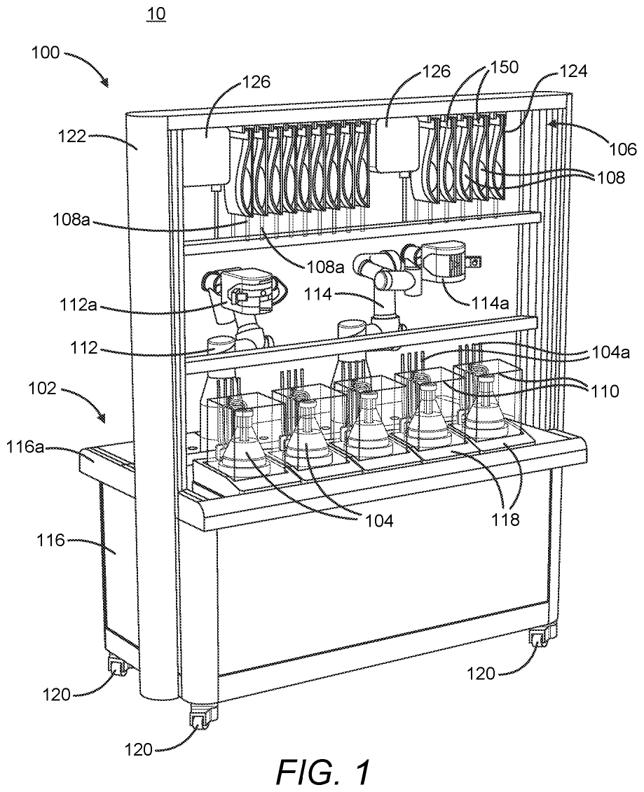

A first embodiment of a closed processing system 10 is shown in FIG. 1, which will now be described. This processing system 10 comprises an automated processing station 100, which in turn comprises an incubation system 102 configured to facilitate the incubation of one or more cell culture chambers 104, and a storage system 106 arranged to facilitate storage of one or more fluid-containing consumables 108. The storage system 106 is in an elevated position relative to the incubation system 102.

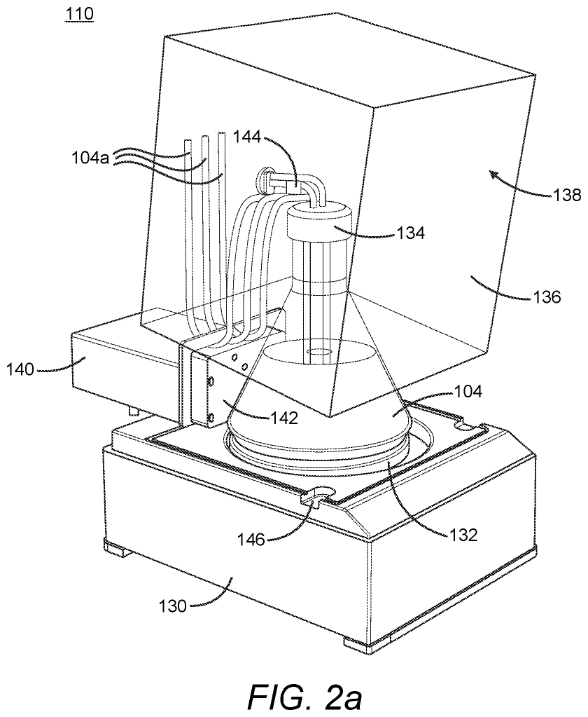

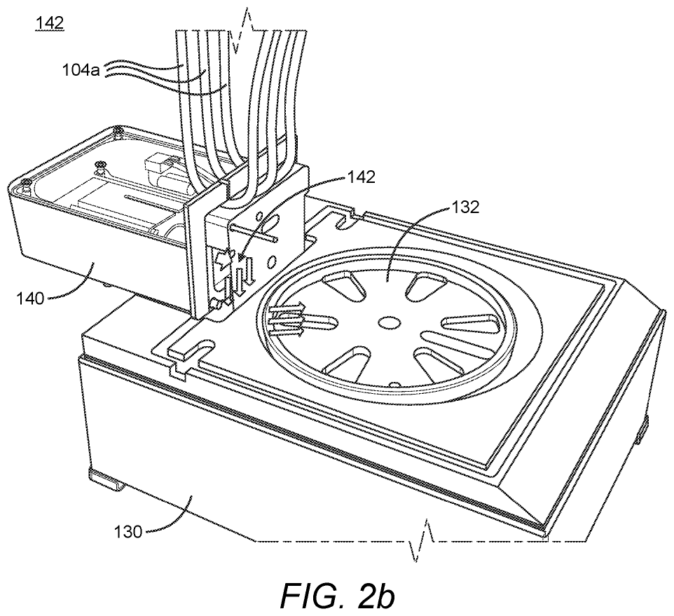

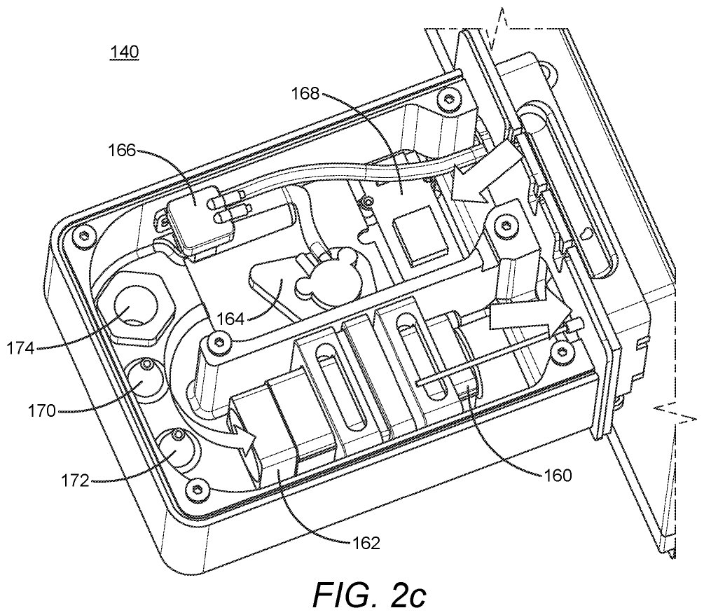

Each cell culture chamber 104 has one or more portions of tube 104a attached, through which fluid can be introduced into the cell culture chamber 104. Such tubes 104a can be seen more visibly in FIG. 2. Similarly, each consumable 108 has a portion of tube 108a attached, through which the contents of the consumable 108 can be extracted. At least one, and preferably both of the tubes 104a, 108a are flexible (i.e. not rigid) tubes.

The incubation system 102 and (consumable) storage system 106 are together arranged at the processing station 100 to enable a robotic device to manipulate a sterile tube weld between a tube connected to a stored consumable 108 and a tube 104a connected to an incubated cell culture chamber 104, as previously discussed. In doing so, an aseptic fluid connection can be created between the cell culture chamber 104 and the consumable 108, thereby maintaining a closed system at the processing station 100.

In this embodiment, the processing station 100 comprises a first robotic device 112 and a second robotic device 114, which together help to automate the processing system 10. The robotic devices 112, 114 are mounted to the processing station 100. The first robotic device 112 comprises a robotic arm having a first end effector 112a configured to perform a sterile tube weld, as generally discussed above. The second robotic device 114 comprises a robotic arm having a second end effector 114a configured as a pumping mechanism, as generally discussed above. Either, or both, of the robotic devices 112, 114 may comprise a tube sealer. Alternatively, a tube sealer may be provided on a separate robotic device.

The processing station 100 comprises a base unit 116 having a support surface 116a provided on an upper side of the base unit 116. The base unit 116 is substantially box-shaped/cube-shaped, having a generally rectangular footprint. The support surface 116a is provided with one or more incubator base 118, which rest on the support surface 116a. Each incubator base 118 is arranged to have mounted thereto an incubation chamber 110 configured to house a cell culture chamber 104. Such an incubation chamber 110 is shown in isolation in FIG. 2, which will be described in more detail further on. The support surface 116a and incubator base(s) 118 therefore form part of the incubation system 102 in this embodiment.

The processing station 100 may comprise a fluid agitation system for agitating fluid contained within an incubated cell culture chamber 110. For example, the incubator base 118 may be configured to agitate an incubation chamber 110 mounted thereon, for example the incubator base 118 may be a rocker plate that rocks the incubation chamber 110 from side to side. Alternatively, or additionally, the incubator base 118 may comprise other means for agitating an incubation chamber 110 mounted thereon, such as an ultrasound source, a vibration source, and/or an orbital shaker. The fluid agitation system may be configured to agitate fluid within the incubated cell culture chamber 110 prior to transfer of fluid from the cell culture chamber to an output container and/or a sample container. In this way, the fluid contained within the output container and/or the sample container will be representative of the contents of the cell culture chamber 110. Preferably, each incubator base 118 comprises a separate means for agitating, thereby allowing different cell culture chambers 110 to be agitated independently such as at different times.

In this embodiment, the robotic devices 112, 114 are also mounted onto the support surface 116a. Furthermore, the base unit 116 is mounted on wheels 120, such as castor wheels, to allow it to be moved and positioned within a bioprocessing system.

A frame 122 extends vertically upward from opposing sides of the base unit 116, to form part of the storage system 106. The frame 122 is substantially rectangular, with a horizontal cross-beam connecting between the upper ends of two opposing vertical members, which are each attached to an opposing side of the base unit 116 to form the frame 122. A support member (or rail) 124 extends at least partway across the width of the frame 122, which is provided for the purpose of supporting the consumables 108 (e.g. a clip or tab 154 provided on each consumable 108 hangs from a groove in the rails) and therefore forms part of the storage system 106. In this way, at least part of the storage system 106 is arranged to be elevated relative to the incubation system 102. Optionally, the storage system 106 may have a portion that is below the incubation system, which may be used to store waste containers. In this way, backflow of waste from the waste containers to the incubation section is inhibited.

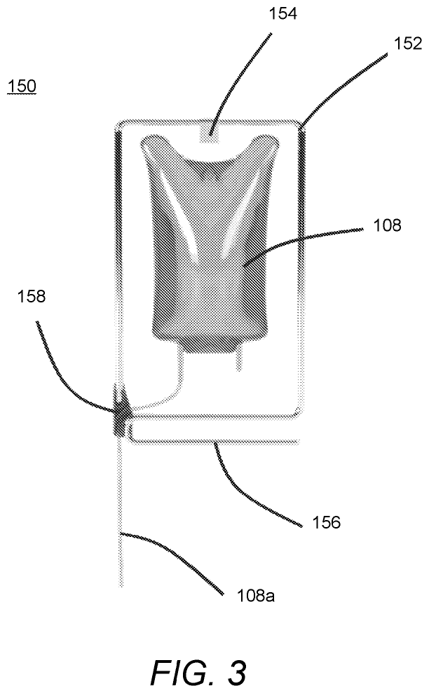

The consumable 108 is stored such that its tube 108a extends beneath the main bag portion (e.g. it remains in situ), such that it can be engaged and/or manipulated by either of the robotic devices 112, 114, for example to create a fluid connection between the consumable 108 and a cell culture chamber 104 held within an incubation chamber 110 that is mounted to an incubator base 118 on the support surface 116a. The consumable 108 may be held by a consumable holder 150, which is configured to be removably mounted to the frame 122/support member 124. An example of such a consumable holder 150 is shown in FIG. 3, which will be described in more detail further on. The tubes 104a, 108a are typically longer than is shown illustratively in FIG. 1, in particular the tube(s) 104a on the cell culture chamber 104.

In this embodiment, the cell culture chambers 104 and various consumables 108 would be loaded onto the processing station 100 by a human operator (i.e. “by hand”) at the beginning of a process, though loading could potentially be automated in the future, for example a by using a separate robotic device in the bioprocessing system configured to perform this function.

A power supply (not shown), a controller/control unit (not shown) and/or a motor drive mechanism (not shown) for each of the robotic devices 112, 114 may be provided in the base unit 116. These components may each be controlled and/or programmed remotely via a computer network (either hardwired or wirelessly) to enable the robotic devices 112, 114 to operate autonomously. The base unit 116 may also house one or more mass flow controllers for gas control. The base unit 116 may be configured to direct temperature-controlled air in or around the consumables 108 in order to keep them refrigerated. Other peripheral power, control and communication componentry may also be provided, which will be well known to the skilled person.

A tube reel 126 may also be mounted to the frame 122, which is configured to deploy similar (weldable) tube that can be used to supplement (e.g. extend) the existing tube 104a, 108a of the cell culture chamber 104 or consumable 108, respectively. For example, in the embodiment shown in FIG. 1, additional tubing from the tube reel 126 can be used to join a consumable 108 to a cell culture chamber 104, or alternatively the tubing 104a and 108a may be configured such their lengths overlap. Optionally, the tube reel 126 may contain lengths of tubing that consists of multiple different materials in sequence (e.g. PVC to Cflex) such that the a cell culture chamber 104 with a portion of tube 104a having an ending made of Cflex can be fluidically connected to a consumable 108 with a portion of tube 108a having an ending made of PVC. Alternatively, or additionally, lengths of tubing (optionally comprising multiple different materials) may be provided on the frame, for example, that the robotic devices can simply grab when required. Preferably, the tubing on the reel and the tubes 104a, 108a of the cell culture chamber 104 and consumable 108 comprise a thermoplastic material.

The tube reel 126 comprises a housing that is mounted to the frame 122, and contains a supply of tube stored wound around a rotatable reel (not shown) inside the housing. Here, there are two such tube reels 126 provided on the frame 122 in a spaced apart configuration. In this way, the robotic devices 112, 114 can avoid handling long flexible lengths of tubing which are challenging for robots to handle. Furthermore, in this way, tubing does not need to be purged when changing between different inputs being pumped into the cell culture chamber 104, but instead the tubing can be replaced.

In this embodiment, each consumable 108 comprises a fluid-containing bag having a portion of flexible tube 108a that allows fluid to be transferred out of the bag. When not fluidly coupled to another tube 104a, an end portion of the consumable tube 108a may be pinched closed to fluidly seal it. To unseal the tube 108a, a portion of the tube 108a towards the bag may be clamped to seal it, and the pinched end portion removed, with the clamped portion released once the tube 108a has been fluid connected to another tube 104a to establish fluid flow. This process may be performed by a robotic device, as previously discussed.

An embodiment of an incubation chamber 110 for incubating a cell culture chamber 104 is shown in FIGS. 2a to 2c, which will now be described. The incubation chamber 110 shown comprises a generally cube-shaped base 130 that is configured to be mounted onto an incubator base 118 that forms part of the incubation system 102. As described above, in this embodiment, the incubator base 118 is provided on the support surface 116a of the base unit 116 of the processing station 100.

The base unit 130 comprises a plate 132 for receiving a cell culture chamber 104. The plate 132 can be seen more clearly in FIG. 2b, i.e. without a cell culture chamber 104 mounted on it. The plate 132 may be configured as a vented offset shaker base 132, for example.

In this embodiment, the cell culture chamber 104 is in the form of a cell culture vessel having a general shape of a conical flask with an opening at its top. A removable cap 134 is (e.g. screw) mounted to the opening of the cell culture chamber 104 to seal its contents from the surrounding environment. A plurality of tubes 104a extend through the cap 134 to allow fluid to be introduced into the cell culture chamber 104, for example from a consumable 108 as described above, while maintaining a closed system within the cell culture chamber 104. This is possible due to the cap 134 sealing around the tubes 104a, the end portions of which are pinched closed to seal them (similar to how is described above for the consumable 108) prior to a robotic device making an aseptic fluid connection via a sterile tube weld, as previously described.

A cover 136 is arranged to fit onto the base 130 to enclose the cell culture chamber 104, i.e. thereby providing a chamber 138 in the incubation chamber 110, within which the cell culture chamber 104 can be incubated. For example, the plate 132 may be heated via a heating element (not shown) located underneath the plate 132. A power supply (not shown) in the base 130 may supply the heating element, for example. In this way, the incubation chamber 110 may be temperature-controlled. Additionally, the incubation chamber 110 may be gas-controlled, where a gas (e.g. oxygen) may be introduced into the incubation chamber 110 via one or more gas ports (see below). A sterilising air filter 144 may also be provided in the chamber 138.

In the embodiment of FIGS. 2a to 2c, heating is achieved by heating air that passes through a heating block 140, which extends into the chamber 138, as shown in FIG. 2c. Arrows indicate air flow through the heating block 140 into the chamber 138, which may contain, a heater 160, a heater fan 162, a CO₂ sensor (board) 164, a CO₂ sensor pump, a thermal sensor (board) 166, and one or more inlets for CO₂ 170, 172 (or another gas mix). One of the inlets 170, 172 may alternatively function as a sensor hole. A cable inlet 174 may be also provided for a supplying power and/or controlling the above-described components in the heating block 140.

The incubator chamber 110 may have one or more openings or gaps for one or more tubes 104a from an enclosed cell culture chamber 104 to pass through the cover 136 of the incubator chamber 110 in a defined orientation. In this way, a user can install a cell culture chamber 104 at the beginning of operation (including clipping in the tubes 104a into the tube routing (not shown), close the cover 136, and then the automated processing station 100 can still manipulate the tube(s) 104a without needing to open the cover 136, and potentially losing temperature control of the chamber 138.

As shown in FIG. 2b, an air pathway 142 is provided to convey air down and around the plate 132. The air pathway is particularly advantageous for a gas permeable cell culture chamber (or “bioreactor”) to ensure air gets underneath the it.

An embodiment of a holder 150 for a consumable 108 is shown in FIG. 3, which will now be described. The consumable holder 150 comprises a substantially rectangular frame 152 within which a consumable (bag) 108 is suspended by a clip/tag 154 mounted on the frame 152. A leg portion 156 extends down from one side of the frame 152, and then across substantially the width of the frame 152, to provide a support for the frame 152, for example if the leg portion 156 is received within a correspondingly sized hole or bore within an apparatus. The frame 152 and leg portion 156 may be formed from a metal material, preferably stainless steel. A tube clip 158 is attached to a side of the frame 152 for retaining a portion of the tube 108a that is fluidly connected to the consumable 108. The tube clip 158 ensures that at least said portion of the tube 108a is retained in a predetermined position, for example so that it can readily found and/or engaged by a robotic device.

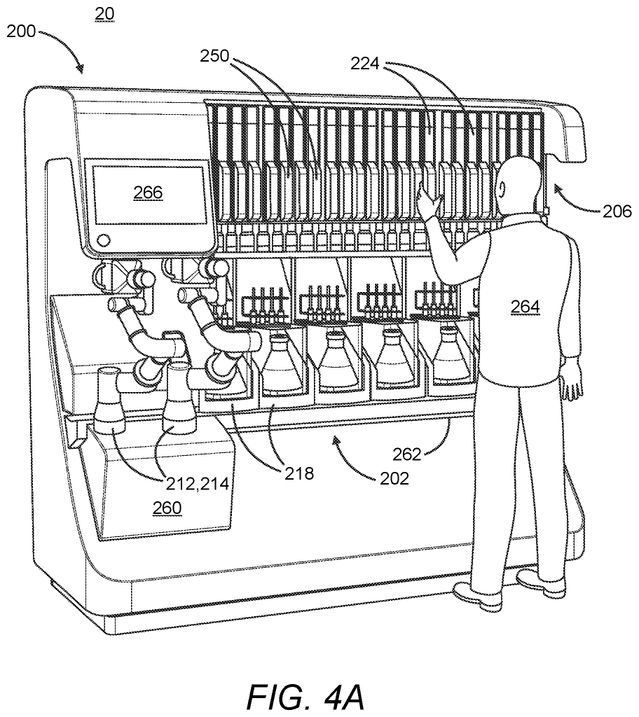

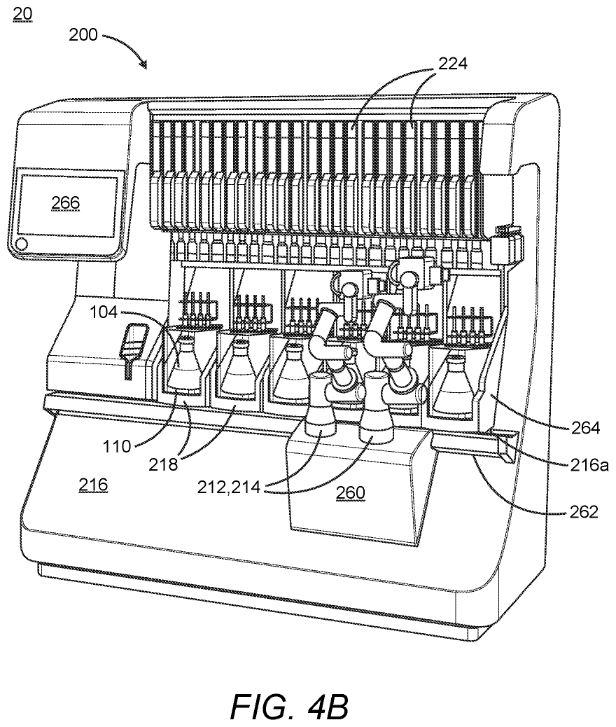

A second embodiment of a closed processing system 20 is shown in FIGS. 4A and 4B, which will now be described.

Similar to the first embodiment, this processing system 20 comprises an automated processing station 200, which in turn comprises an incubation system 202 configured to facilitate the incubation of one or more cell culture chambers 204, and a storage system 206 arranged to facilitate storage of one or more fluid-containing consumables 208. The storage system 206 is in an elevated position relative to the incubation system 202.

The incubation system 202 and (consumable) storage system 206 are together arranged at the processing station 200 to enable a robotic device to manipulate a sterile tube weld between a tube 208a connected to a stored consumable 208 and a tube 204a connected to an incubated cell culture chamber 204, as previously discussed.

Similar to the first embodiment, the processing station 200 comprises a first robotic device 212 and a second robotic device 214, which help to automate the processing system 20. In this embodiment, however, the robotic devices 212, 214 are mounted to a movement system comprising a moveable platform 260 that is arranged to move relative to the processing station 200. The processing station 200 comprises a base unit 216 having a support surface 216a. One or more incubator base 218 are located on the support surface 216a. The moveable platform 260 is mounted to a rail system 262, comprising one or more rails (not shown) which extends along an underside of a support surface 216a of the base unit 216. Thus, more specifically, the moveable platform 260 (and hence the robotic devices 212, 214 mounted to it) are arranged such it can move relative to the incubator base(s) 218, such that the robotic devices 212, 214 can attend each incubator base 218 individually when required.

As with the first embodiment, each incubator base 218 is arranged to have mounted thereto, thereon or therein an incubation chamber 110 configured to house a cell culture chamber 104, as shown in FIG. 2. Similarly, the support surface 216a and incubator base(s) 218 form part of the incubation system 202 in this embodiment. Each incubator base 218 is isolated from its neighbour by a partition wall 264.

The first and second robotic devices 212, 214 may comprises robotic arms with end effectors attached for manipulating tubes, as previously discussed for the first embodiment.

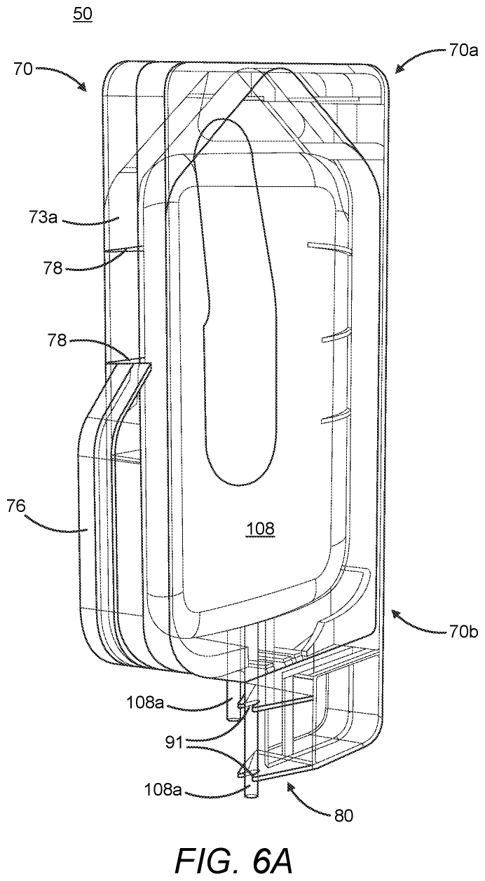

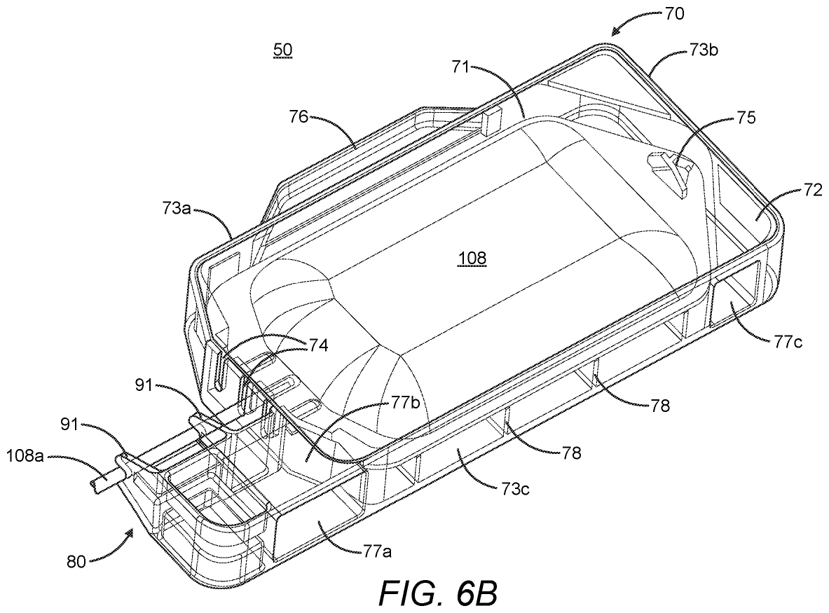

In this embodiment, the storage system 206 comprises a plurality of slots 224 configured to receive and store a consumable 108. The consumable 108 may be held within a cartridge 50 that is configured to be installed into the slots 224 by a human operator, as shown. Such a consumable cartridge 50 is shown in FIGS. 6A and 6B, which will be discussed in more detail further on.

The consumable 108 is stored such that its tube 108a extends beneath the main bag portion of the consumable 108, such that it can be engaged and/or manipulated by either of the robotic devices 212, 214, for example to create a fluid connection between the consumable 108 and a cell culture chamber 104 held within an isolation chamber 210 that is mounted to an incubator base 218 on the support surface 216a.

A user interface 266 is provided to control autonomous operation of the moveable platform 260 and robotic devices 212, 214. A power supply (not shown), a control unit (not shown) and/or a drive mechanism (not shown) for the moveable platform 260 and robotic devices 212, 214 may each be provided in the base unit 216. These components may each be in communication with the user interface 266, which may comprise a touch screen or similar programmable computer device, to enable the robotic devices 212, 214 to operate autonomously. In addition, or alternatively, one or more of these components may controlled and/or programmed remotely via a computer network (either hardwired or wirelessly) to enable the robotic devices 212, 214 to operate autonomously. Other peripheral power, control and/or communication componentry may also be provided, which will be well known to the skilled person.

Other peripheral power, control and communication componentry may also be provided, which will be well known to the skilled person.

In this embodiment, everything is front facing so that it can easily be wipe-down cleanable. Individual incubation chambers 110 may be spaced apart with some separation, and preferably also a partition wall 264, between them such that if there is a leak from an individual incubation chamber 110, the area can be fully cleaned while other incubation chambers 110 remain in operation.

Having two robot devices 212, 214 enables complicated tasks to be performed in parallel, for example some tubes 104a, 108a may be manipulated by the first robotic device 212, while the second robotic device 214 engages with fluidly connected tubes 104a, 108a to pump fluid between a consumable 108 and a cell culture chamber 104. Furthermore, in the event that there is a weld failure, the second robotic device may be able to fluidically seal the tubing upstream and downstream of the weld, enabling a t (or T) weld to be performed By mounting the robotic devices 212, 214 on a moveable platform 260 mounted to a rail system 262, it may be possible to share the robotic devices 212, 214 with other processing stations or modules within a larger system. In another example (not shown), the robotic device may be directly driven on the rail system 262 (e.g. using a linear rail or similar), or alternatively a rail may simply act as a guide and the robotic device may be a mobile robot running on wheels, with the robot hooked on the rail. This latter option is potentially advantageous as it may be easier to daisy chain more systems together, while using the same robot, but not have to deal with the complexity of a fully autonomous robot.

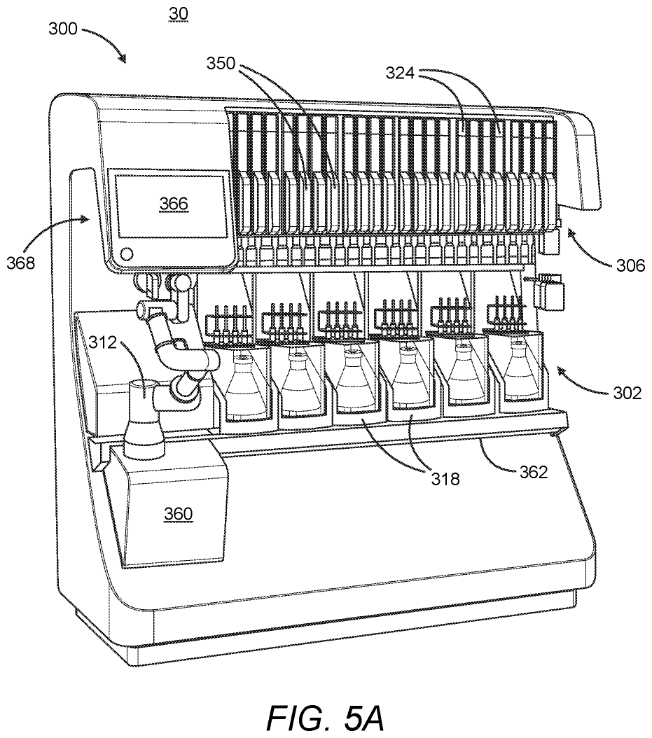

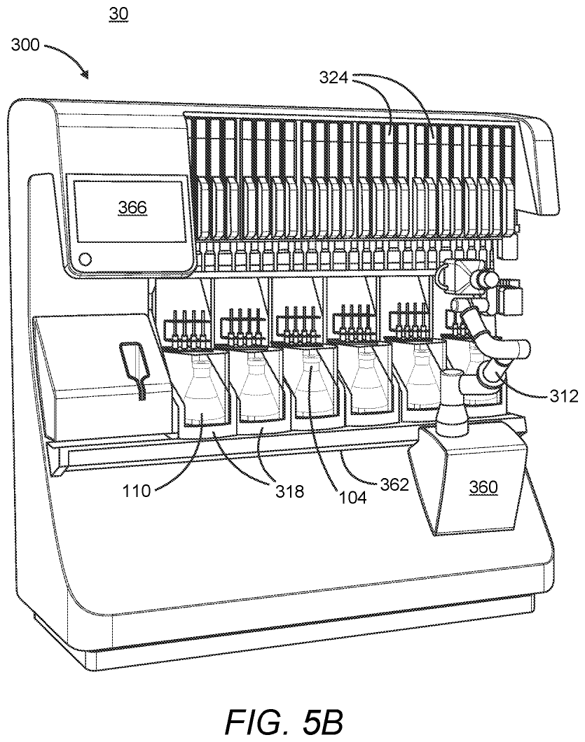

A third embodiment of a closed processing system 30 is shown in FIG. 5, which will now be described. The processing system 30 comprises an automated processing station 300 that is similar to the automated processing station 200 of the second embodiment (previously described) in all aspects, with the exception that with this processing station 300 a single robotic device 312 is provided on a moveable platform 360, which helps to automate the processing system 30. In this embodiment, the robotic device 312 is configured to have interchangeable end effectors (not shown), which are stored in an interchange station 368 located behind the user interface 366. A robot tool changer (not shown) may be hidden behind the user interface 366, and the robotic device 312 may select an appropriate tool depending on need. The end effectors may include a tube welder, a peristaltic pump, a tube sealer and/or a tube disconnector (e.g. a heated cutting element, such as a blade or wire). Advantageously, this “single robotic device” arrangement should be cheaper than the processing station 200 of the second embodiment having “multiple robotic devices” due to cost of robotic devices. Also, a single robotic device 312 would take up less space, so the moveable platform 360 can be smaller that would be required for two robotic devices.

An exemplary embodiment of a cartridge 50 (or “device”) for holding a consumable 108 is shown in FIGS. 6A and 6B. In the example shown in FIG. 6A, the consumable 108 is connected to two flexible tubes 108a. Consumables 108 may be pre-installed into such cartridges 50, which are configured to hold the tube(s) 108a in a defined orientation. The cartridge 50 comprises a housing 70 configured to hold the consumable 10; in other words, the housing 70 provides a first portion of the cartridge 50 for holding the consumable 108. The cartridge 50 may also provide insulation and/or heat conduction with the consumable 108, such that temperature of each consumable 108 can be individually adjusted.

An upper surface and lower surface may be provided on the housing 70 separated by a pair of opposing side walls 73a, 73c (i.e. a first and second side wall 73a, 73c), and at least one end wall 73b, thereby forming a cuboidal shape. The housing 70 may have a longitudinal axis with a first end 70a and a second end 70b.

The housing 70 may comprise a tray 70 with a cavity 71 shaped to receive the consumable 108 therein. The upper surface of the housing 70 may be a removable cover 72 that substantially encloses the consumable 108 within the cavity 71; in this way, the consumable 108 may be conveniently added and/or removed from the cartridge 50. Furthermore, this means that the cartridge 50 has a rigid outer surface that may protect the consumable 108 and may enable the cartridge 50 to be reliably installed into the storage system 206, 306 of a processing station 200, 300, such as into the slots 224, 324.

Preferably, the cavity 71 has dimensions comparable to those of the consumable 108 such that the consumable 108 fits tightly into the cavity 71, thereby holding the consumable 108 in position. This reduces the flexibility of the consumable 108 by retaining the consumable 108 in a given shape. The housing 70 may be rigid and may be made of any suitable material, such as plastic or metal. In other examples, the housing 70 may comprise a material that has some flex, such as any material with elastic properties. In this way, the consumable 108 may be forced into the cavity 71 by stretching the cavity 71 slightly upon insertion. The housing 70 will then flex back into its original shape once the consumable 108 is inserted, thereby securing the consumable 108.

Advantageously, use of the cartridge 50 to house a consumable 108 comprising a bag prevents the bag from bulging outwards when full. The housing 70 may also contain external insulation, and routing for internal cold airflow, such that a consumable contained within the housing 70 can be kept at a specified temperature. A thermistor (not shown) may also be inbuilt to the housing 70 to monitor temperature.

The housing 70 may comprise at least one clamp 75 to hold the consumable 108 inside the cavity 71. In this example, the housing 70 has a clamp 75 in the cavity 71 towards the first end 70a of the housing 70. The clamp 75 may be a tab or a hook. The consumable 108 may be mounted via a hanging aperture provided in the bag of the consumable 108.

The housing 70 may comprise a means for engagement 76 provided on an external surface of the housing 70. In this example, the means for engagement 76 is a handle 76 attached to the first side wall 73a. The handle 76 may allow the cartridge 50 to be manipulated and moved by a human operator and/or a robotic device.

For example, the handle 76 may allow the cartridge 50 to be carried and installed into the storage system 206, 306 of a processing station 200, 300, such as into the slots 224, 324.

One or more ribs 78 are provided around the perimeter of the housing 70, extending between upper and lower surfaces of the housing 70. An identification mark, such as a barcode, QR code, RFID or NFC code may be provided on the cartridge 50 to allow the cartridge 50 to be loaded easily into the storage system 206 in a plug/play manner and/or to allow the cartridge 50 be identified and tracked. For example, the cartridges 50 may be automatically identified when inserted into a slot 224, 324 of the storage system 206, 306.

The housing 70 may comprise at least one recess (e.g. gap or opening) 74 in an end portion of the housing (e.g. the second end 70b), through which the tube 108a may pass. The recess 74 is preferably aligned with the portion of the consumable 108 that connects to the flexible tube 108a. There may be a plurality of recesses 74 present on the housing 70 for when the consumable 108 is fluidly connected to more than one tube 108a.

The cartridge 50 comprises a second portion 80 configured to retain the flexible tube 108a connected to the consumable 108. The second portion 80 may extend from the second end 70b of the housing 70 and is preferably located adjacent to the recesses 74. The second portion 80 may comprise a plurality of tube retaining elements 81 (e.g. tube clips) to retain the flexible tube 108a at a plurality of positions along a predetermined path. In this example, the second portion 80 has a pair of tube retaining elements 81, the tube retaining elements 81 being spaced apart such that a portion of the tube 108a may be held between them in a substantially taut manner to facilitate engagement by a robotic device. In this way, a robotic device may engage the tube 108a at a position between the pair of tube retaining elements 81.

The tube 108a may be secured to each of the tube retaining elements 81 by exerting a force on the tube 108a so as to push the tube 108a into the tube retaining element 81, resulting in the tube retaining elements 81 securing the tube 108a at a given position. The tube retaining elements 81 may alternatively comprise any suitable means for coupling the portion of tube 108a to the second portion 80 such as a hook or a clasp. The portion of tube 108a retained by the tube retaining elements 81 may be permanently retained or may be removably secured.

The cartridge 50 may comprise a means for cooling 77 the consumable 108 held within the housing 70. In this example, the means for cooling 77 is at least one air port 77 (or “air duct”) in the first portion 70 and/or the second portion 80 of the cartridge 50; in this way, cool air may be supplied to the at least one air port 77 in order to cool the consumable 108. Specifically, the cartridge 50 comprises a first external air port 77a in communication with an internal air port 77b to facilitate the introduction of air into/out of the cavity 71. A second external air port 77c in fluid connection with the cavity 71 is provided in the housing 70 to allow air to flow out of/into the housing 70. Alternatively, a Peltier and fan could be built within the consumable (not shown).

A single incubator base may be provided on the support surface, configured to have mounted thereon a single incubation chamber or multiple incubation chambers. Rather than each incubation chamber being configured to contain a single cell culture chamber, a single incubation chamber may be configured to contain (and hence incubate) multiple cell culture chambers.

The processing station may work with multiple (e.g. patient) samples at once, or it may instead distribute the contents of a single (e.g. patient) sample across multiple cell culture chambers (e.g. expansion chambers) in order to carry out a design of experiments on that chamber adjusting conditions such as feeding times/rates, reagent types, temperature, pH etc. Where the processing station includes a cell analyser (e.g., a “cell analysis unit” or similar means), the incubation chamber (bioreactor) conditions or parameters (i.e. feed rates etc) may be automatically adjusted in response to a measured parameter (e.g. cell number). The cell analyser may be a cell counter, a cytometer, or any other means of cell analysis or media analysis.

As a further alternative, the pumping (and or valving) means may be provided as a separate device or apparatus on the processing station (potentially one pumping means per incubation chamber) and the robotic device may insert tubing forming a fluid connection into the pumping means. In this way, the control of fluid can be maintained while the robotic device is tending other incubation chambers.

While the processing stations illustrated in the accompanying figures show the consumables (e.g. media/reagent bags) stored at room temperature within the storage system, a section of the storage system may instead be configured to refrigerate the consumables. This may be achieved, for example, by blowing cold air through the air duct of each consumable (allowing individual control as to the temperature of each consumable by varying the amount of air going through each one), or by blowing cold air around all of the consumables (akin to a refrigerator in a supermarket with an open door, or a sliding door).

The processing station may transfer samples between different cell culture chambers (e.g. that are already pre-loaded onto the processing station). For example, it may be desirable to perform activation in one chamber and transfection/expansion in a subsequent chamber that has been coated with retronectin. In addition, or alternatively, it may be desirable to begin expansion in a small chamber before transferring the culture to a larger chamber. The process or operation performed at a processing station may be “reconfigurable” in the sense that a human operator may program a sequence of sampling, media changes, when harvest should occur, etc, for a particular use, and then reprogram the processing station for another use.

The robotic devices may also have probes that are capable of interrogating key analytes on independent cell culture chambers, enabling at line monitoring of analytics. For example, a robotic device may carry a probe for Raman spectroscopy (to sense metabolites like Glucose or Lactate) or an optical probe for fluorescence lifetime (to measure pH and CO₂ using a “presens” sensor) or microscope (to measure cell morphology in adherent cell cultures) and move the probe between each cell culture chamber. Such an approach may be advantageous as the cost of the Raman system can be amortised across multiple patient samples. It will be appreciated that such probes are not required to directly contact the cells in order to collect data, thereby allowing the cells to be monitored while maintaining the closed system.

The processing systems described herein may further comprise one or more cameras or sensors, such as a machine vision system, that enable all the consumables 108 to be tracked throughout a particular process. In other words, the systems may comprise means for maintaining traceability of the consumables 108. The one or more cameras or sensors may be located on the robotic devices. The one or more cameras or sensors may be configured to identify an identification mark on at least one of the consumables 108, cell culture chambers 104, and the tubes 104a, 108a. The one or more cameras or sensors may be optical so as to detect bar codes and/or QR codes, or may be non-optical so as to detect RFID or NFC tags.

A bioprocessing system comprising one or more such processing stations is easy to adopt and easy to scale. For example, the system may initially have a single processing station, but additional processing stations may be added as requirements grow. A fully automated system may be achievable by incorporating multiple processing stations, optionally wherein the robotic devices are not provided on the processing station but are instead provided on autonomous mobile “manipulation” units/mobile robotic devices that can attend multiple processing stations, when required.

A bioprocessing system may therefore incorporate multiple processing stations, as described herein, running in parallel, each processing station comprising: means to aseptically connect a consumable (e.g. cell bag) to an expansion chamber (e.g. a cell culture chamber) and transfer cells to the expansion chamber (i.e. seed the cells); means to aseptically connect a different consumable (e.g. media bag) to the expansion chamber and transfer fluid to the expansion chamber (i.e. feed the cells); means to agitate the expansion chamber (e.g. so that the sample is representative) and to take samples from the expansion chamber; means to aseptically connect another consumable (e.g. an output bag) to the expansion chamber and transfer the fluid to the output bag (i.e. harvest cells)—agitating the expansion chamber prior to the transfer to ensure all cells are distributed; means to disconnect the consumables (e.g. cell bags, media bags and the output bags); and means for controlling an automated sequence of operation.

The means for controlling an automated sequence of operations may be provided by a processing and control unit (not shown). The processing and control unit may be part of the closed system or may be part of the bioprocessing system as a whole. The processing and control unit may be used to control multiple closed systems in parallel. The automated sequence of operation may be controlled according to one or more predetermined workflow(s), preferably one or more reconfigurable workflows. In this way, the particular method being performed by the bioprocessing system may be readily modified or adapted without requiring modification to the bioprocessing system itself.

The means for controlling an automated sequence of operation may be configured to automatically schedule a sequence of actions to be followed by the closed system. The sequence of actions may be automatically updated based on inputs received from at least one sensor of the bioprocessing system and/or the closed system. In this way, multiple cell culture chambers may be processed simultaneously by the closed system while minimizing the risk of conflicts between their corresponding workflows. For example, the sequence of operations may be scheduled to minimize and preferably prevent any of the robotic devices or other parts of the closed system or bioprocessing system being required simultaneously for separate workflows. If it is not possible to avoid a conflict, the means for controlling an automated sequence of operations may delay one of the conflicting actions based on a pre-programmed or user-configurable list of priorities.

The means for controlling an automated sequence of operation may be configured to simulate the automated sequence of operation prior to the bioprocessing system performing said sequence. The means for controlling an automated sequence of operation may convey at least one outcome of the simulation to an operator. The at least one outcome may comprise: an indication when particular operations occur, an indication when manual steps may need to be performed, and/or an indication that a conflict between two concurrent operations may (or may not) occur. The bioprocessing system may further comprise a monitoring system to verify that the automated sequence of operation has occurred. The monitoring system may be provided by the means for controlling the automated sequence of operation of the processing station.

The processing system may comprise a cell analyser (e.g., “cell analysis unit”) such as a cell counter/flow cytometer (or some other means of cell analysis/media analysis) and means for transferring samples from the cell culture chamber into the cell analyser. For example, a cytometer station configured to hold an analysis chamber (or analysis unit) may be mounted to the support surface of the base unit. The analysis chamber may have a flexible tube connected thereto. The tube welder may form a tube weld between the corresponding tubes of the cell culture chamber and the analysis chamber, and the peristaltic pump may transfer a sample of the contents of the cell culture chamber into the analysis chamber for analysis. Depending on the measurements taken by the cell analyser, one or more processing parameters of the closed system may be adjusted. For example, the temperature of the incubation chamber may be changed, and/or the feed rates into the incubation chamber may be changed.

It is also possible to connect aseptically a waste bag (i.e. consumable) to the cell culture chamber and remove waste media from the top of the cell culture chamber (i.e. reduce media volume without removing cells, either for the purposes of concentration or media exchange) at the processing station.

Advantageously, the processing station may be capable of carrying out each of the following steps of a cell therapy process: activation, transfection, and expansion, at a single location (e.g. at a standalone station). Furthermore, easy process development may be possible due to the ability to carry out parallel processes simultaneous with automatic sampling and cytometry. Due to the automation of the processing station, samples may be taken 24 hours a day, every day. In addition, the processing station may automatically maintain traceability between consumables (e.g. bags) and cell culture chambers (i.e. expansion chambers) in a space efficient form factor.

While the foregoing is directed to exemplary embodiments of the present invention, it will be understood that the present invention is described herein purely by way of example, and modifications of detail can be made within the scope of the invention. Furthermore, one skilled in the art will understand that the present invention may not be limited by the embodiments disclosed herein, or to any details shown in the accompanying figures that are not described in detail herein or defined in the claims. Indeed, such superfluous features may be removed from the figures without prejudice to the present invention.

Moreover, other and further embodiments of the invention will be apparent to those skilled in the art from consideration of the specification and may be devised without departing from the basic scope thereof, which is determined by the claims that follow.

1. An automated system for performing bioprocessing, comprising:
an incubation system configured to accommodate at least one incubation chamber for incubating a cell culture chamber having a flexible tube connected fluidly thereto; and
a storage system configured to store at least one fluid-containing consumable having a tube connected fluidly thereto;
wherein the incubation system and storage system are together arranged to enable a robotic device to manipulate a tube weld between said flexible tube of the incubated cell culture chamber and said tube of the stored consumable, whereby said tube weld forms an aseptic fluid connection between the cell culture chamber and the consumable such that a closed system is maintained.

2. (canceled)

3. The system of claim 1, wherein the incubation system is further configured to store in each incubation chamber a cell culture chamber with its flexible tube in a predetermined orientation.

4. (canceled)

5. (canceled)

6. (canceled)

7. The system of claim 1, further comprising a cell analysis unit, wherein the at least one robotic device is further configured to manipulate a fluid connection between the cell analysis unit and an incubated cell culture chamber.

8. (canceled)

9. (canceled)

10. The system of claim 1, further comprising an automated processing station configured as a stand-alone processing station, including a support platform configured to form part of the incubation system.

11. The system of claim 10, wherein at least one robotic device is also provided on the processing station.

12. (canceled)

13. A bioprocessing system comprising:
the automated system of claim 10, and
a separate robotic device configured to move independently relative to the automated processing station.

14. The system of claim 11, wherein the robotic device is configured to manipulate the fluid connection whereby to create a tube weld between the flexible tube of an incubated cell culture chamber and the flexible tube of a stored consumable for the transfer of fluid therethrough, said tube weld being a sterile tube weld formed between free ends of the respective tubes.

15. The system of claim 11, wherein the robotic device is configured to manipulate the fluid connection whereby to disconnect the flexible tube of an incubated cell culture chamber and the flexible tube of a stored consumable.

16. (canceled)

17. The system of claim 11, wherein the robotic device is configured to manipulate the fluid connection whereby to pump fluid between a consumable and an incubated cell culture chamber via said fluid connection.

18. (canceled)

19. The system of claim 11, wherein the robotic device is configured to manipulate the fluid connection whereby to engage and/or position the respective at least one flexible tube of an incubated cell culture chamber and at least one flexible tube of a stored consumable relative to one another to form the fluid connection.

20. The system of claim 11, wherein the at least one robotic device is further configured to manipulate a fluid connection between a first incubated cell culture chamber and a second incubated cell culture chamber.

21. The system of claim 11, wherein the robotic device is configured to use interchangeable end effectors, the system further comprising an end effector storage system for storing one or more interchangeable end effectors for use by the robotic device.

22. The system of claim 11, wherein the robotic device comprises an end effector on a robotic arm and the robotic device further comprises at least one of a probe or sensor located on the end effector.

23. The system of claim 1, further comprising a controller for controlling a sequence of automated operations of the system.

24. (canceled)

25. (canceled)

26. (canceled)

27. The system of claim 1, further comprising a fluid agitation system for agitating fluid contained within an incubated cell culture chamber.

28. (canceled)

29. (canceled)

30. The system of claim 1, wherein the storage system comprises a refrigeration or heating unit for regulating the temperature of one or more consumable.

31. The system of claim 1, further comprising a tube supply device arranged to provide supplementary tubing for at least one of a cell culture chamber or a consumable, preferably wherein the device comprises a reel of tube supply.

32. The system of claim 1, further comprising means for identifying an identification mark on at least one of the following: consumables, tubes, cell culture chambers.

33. (canceled)

34. A bioprocessing system comprising a plurality of automated systems according to claim 1.

35. A method comprising performing bioprocessing using a system according to claim 1.
